(19) United States
(12) Patent Application Publication
(10) Pub. No.: US 2024/0352533 A1
(43) Pub. Date: Oct. 24, 2024

(54) Monocarboxylic Acid Transporter 4 as a Biomarker for Hypoxia Driven Tumor Growth and Treatment

(71) Applicant: NantBio, Inc., Culver City, CA (US)

(72) Inventor: Paul Weingarten, Culver City, CA (US)

(73) Assignee: NantBio, Inc., Culver City, CA (US)

(21) Appl. No.: 18/643,905

(22) Filed: Apr. 23, 2024

Related U.S. Application Data
(60) Provisional application No. 63/529,532, filed on Jul. 28, 2023.
(60) Provisional application No. 63/461,522, filed on Apr. 24, 2023.

(51) Int. Cl. C12Q 1/6886; G01N 33/574

(52) U.S. Cl. CPC C12Q 1/6886; G01N 33/57484; C12Q 2600/106; C12Q 2600/158

(57) ABSTRACT

Compositions and methods for treatment of tumor cells are presented in which tumor cells that overexpress monocarboxylate transporter 4 (Mct-4), HIF-1α, Hexokinase-2, and/or CD147 are targeted with one or more metabolic inhibitors.

This application claims priority to and the benefit of U.S. Provisional Application No. 63/461,522 (filed on Apr. 24, 2023) and 63/529,532 (filed on Jul. 28, 2023), the entire content of which is incorporated herein by reference.

### FIELD OF THE INVENTION

The field of the invention is compositions and methods of cancer treatment, especially as it relates to treatment of tumor cells expressing high levels of monocarboxylate transporter 4 (Mct-4).

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Most tumor cells have significantly increased anabolic activity and typically make use of the glycolytic pathway to provide ATP for synthesis of numerous metabolites required for the rapid growth. With increased glycolysis, intracellular levels of lactate increase due to high levels of pyruvate (which is the product of glycolysis). As such, the tumor cells need to clear excessive lactate to avoid intracellular acidification, and lactate homeostasis is typically maintained by a number of transmembrane monocarboxylate transporters (MCTs; collectively known as SLC16a transporter family). Among these MCTs, only a subset (MCT1, MCT2, MCT3 and MCT4) will export small monocarboxylates such as lactate, pyruvate, and ketone bodies (acetoacetate and β-hydroxybutyrate).

More recently, expression analyses have established that most aggressive tumor types express markedly elevated levels of MCT1, MCT4, or both, and that CD147 is required for MCT1 and MCT4 cell surface expression. Notably, it was demonstrated that blocking lactate export by inhibiting the myc target MCT1 disabled glycolysis and glutathione synthesis (see Cancer Res. 2014, 74, 908-920; Proc. Natl. Acad. Sci. USA 2011, 108, 16663-16668).

The expression of MCT1 and MCT4 is regulated by two major oncogenic transcription factors, MYC and hypoxia inducible factor-1α (HIF-1α), respectively, that direct marked increases in the production of key proteins that support aerobic glycolysis, including amino acid transporters and enzymes involved in the catabolism of glutamine and glucose. Indeed, HIF-1α mediates activation of over 100 genes involved in adaptation to hypoxia (e.g., genes involved in glucose metabolism, proliferation, migration, angiogenesis, and metastasis). Viewed from a different perspective, HIF-1α mediates a shift to aerobic glycolysis. FIG. 1 is an exemplary schematic illustration of selected HIF-1α mediated effects precipitated by hypoxia in a cell.

Notably, in many human tumors MCT1 and MCT4 are inversely expressed. Malignancies having MYC involvement and hypoxic tumors are generally resistant to current frontline therapies, with high rates of treatment failure, relapse, and high patient mortality. While some MCT inhibitors are known in the art, most of these are weak MCT inhibitors (i.e., effective at high micromolar levels), such as α-cyano-4-hydroxycinnamate, stilbene disulfonates, phloretin, and related flavonoids as well as coumarin-derived covalent MCT inhibitors. Unfortunately, due to their low affinity, therapeutic effect in cancer therapy remained elusive. A highly potent MCT inhibitor was more recently identified via a cell-based assay seeking immunosuppressive agents that inhibit NFAT1-directed IL-2 transcription (J. Med. Chem. 1995, 38, 14, 2557-2569), and MCT1 inhibition as its mechanism of action was described a full decade later (Nat. Chem. Biol. 2005, 1, 371-376). However, MCT1 inhibitors are unlikely to be effective for MCT4 inhibition as MCT4 expression is low when MCT1 expression is high.

Thus, even though various compositions and methods of cancer treatment are known in the art, all or almost all of them suffer from several drawbacks. Therefore, there remains a need for improved cancer treatments.

### SUMMARY OF THE INVENTION

The inventive subject matter is directed to various compositions and methods of treatment of cancer in which a metabolic inhibitor, and especially an inhibitor that interferes with Mct4, is administered upon determination that Mct4 expression is elevated relative to corresponding non-tumor tissue.

In one aspect of the inventive subject matter, the inventors contemplate a method of predicting therapeutic efficacy of metabolic inhibitors in the treatment of cancer that includes a step of performing quantitative assays for tumor levels of monocarboxylate transporter 4 (Mct4), HIF-1a, Hexokinase-2, and/or CD147, and another step of administration of a metabolic inhibitor when the tumor expression of the marker is significantly greater than its expression in healthy tissue. The administration step is typically conducted when the marker has at least 10% or at least 20% or at least 30% higher expression in a tumor cell compared to its healthy counterpart.

In some embodiments, the metabolic inhibitor is a HIF-1a inhibitor (e.g., a mitochondrial targeted inhibitor and/or an inhibitor of HIF-1a interaction with P300). In further embodiments, it is contemplated that quantitative assays of the tumor levels of metabolic proteins comprising Mct4, HIF-1a, HK2, and/or CD147 are compared with the same protein levels assayed in normal, non-cancerous tissue. Most typically, but not necessarily, the assay measures tumor protein levels, or tumor protein levels and normal tissue protein levels. Alternatively, or additionally, the assays may also measure tumor mRNA levels, or tumor mRNA levels and normal tissue mRNA levels. Where desired, the assays may also measure tumor metabolic flux.

In another aspect of the inventive subject matter, the inventor also contemplates a composition for use in the treatment of solid tumors, and especially contemplated compositions comprise 2-deoxyglucose and an inhibitor of HIF-1a. Therefore, and viewed form a different perspective, the inventor also contemplates a composition for use in the treatment of solid tumors, the composition comprising a means of binding to Mct4, wherein the means, when bound to Mct4, inhibits growth of Mct4-expressing cells in an in vitro tumorsphere model of cell growth. Among other suitable options, the means can be an antibody or binding domain thereof, and/or the means can be a T cell or a natural killer (NK) cell, wherein the T cell or NK cell comprises a CAR, and wherein the CAR comprises the means for binding Mct4.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is an exemplary schematic illustration of selected effects precipitated by hypoxia in a cell.

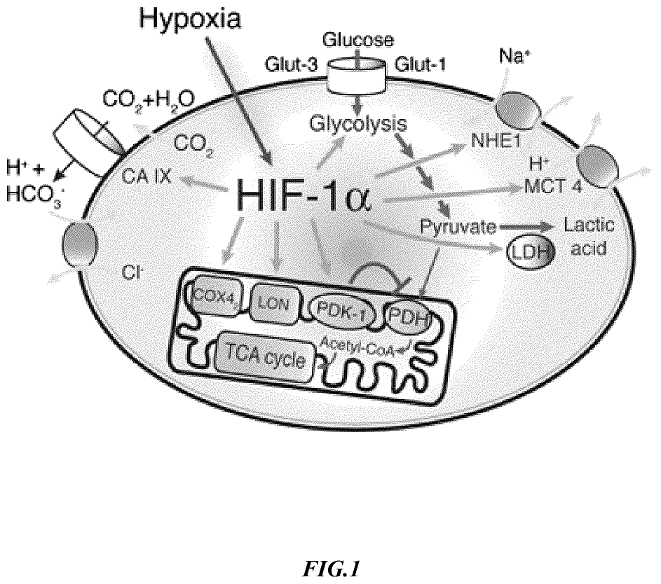

FIG. 2 is an exemplary schematic illustration of HIF pathways and potential points of interference.

FIG. 3 is a graph depicting exemplary results for HIF-1a activity in cells exposed to NTW-4975, and cell viability, tumor sphere growth, and attached growth inhibition of cells so treated.

FIG. 4 depicts exemplary results for inhibition of selected genes in Ishikawa cells by NTW-4975.

FIG. 5 is a table depicting results for 72-hr viability assay in human hepatocytes.

FIG. 6 is a graph depicting exemplary data for inhibition of HIF-dependent gene expression by NTW-4975.

FIG. 7 are graphs depicting exemplary results for tumorsphere volume decrease in Mct4 depleted cells.

FIG. 8 are photographs of Northern blots depicting exemplary results for Mct4 associated CD147 co-expression in stable transfected clones.

FIG. 9 are graphs depicting exemplary results for tumorsphere volume as a function of Mct4 overexpression and cell viability (as determined by ATP assay).

FIG. 10 is a graph depicting exemplary results for reversal of HIF-1a inhibitor based tumorsphere growth by Mct4 overexpression.

FIG. 11 is a graph and Western blot depicting inhibition of TGF-b driven invasion of Panc1 cells by siRNA-mediated depletion of Mct4.

FIG. 12 depicts Western blots and associated graphs indicating that Rab25, HIF-1a, Mct4, and CD147 expression increases in 3D growth format for both HCT116 and Ishikawa cells.

FIG. 13 is a table showing distinct mRNA transcription between 2D growth under hypoxia vs. 3D growth under normoxic conditions.

FIG. 14 is a graph showing increased Glut-1 & Glut-3 (Glucose Transporters) mRNA transcription between 2D growth vs. 3D growth, both under normoxic conditions.

### DETAILED DESCRIPTION

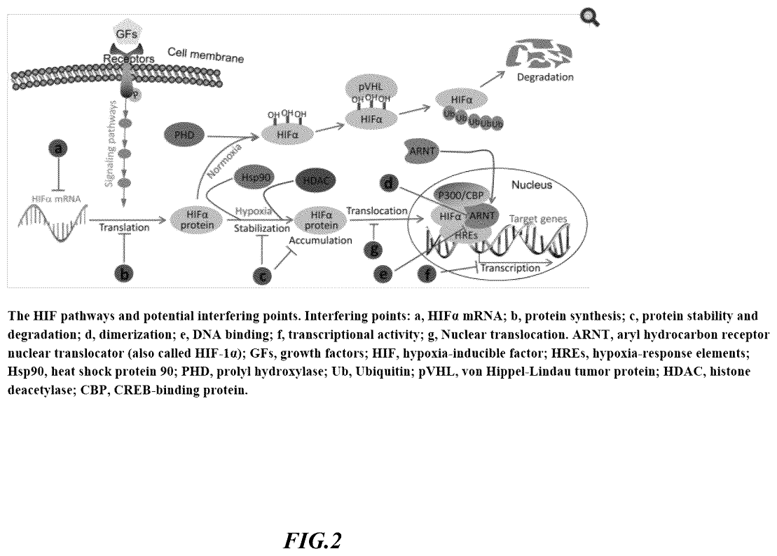

Since tumor cells are frequently hypoxic, the cell's various response mechanisms to hypoxic conditions, and especially HIF-1 controlled pathways, present possible pathways for cancer therapy. FIG. 2 is a schematic illustration of HIF exemplary pathways and potential points of interference. Based on these pathways, downstream elements regulated by these pathways, and other considerations, the inventor has discovered that increased expression (at least 10%, or at least 20%, or at least 30%, or at least 40%, or at least 50% higher than a matched normal cell) or presence of selected molecular markers, and especially monocarboxylate transporter 4 (Mct4), HIF-1α, Hexokinase-2, and CD147, is predictive for likely therapeutic effect of metabolic inhibitors in the treatment of cancer.

In particular, the inventor discovered that certain HIF-1α inhibitors (e.g., NTW-4975) had significant inhibitory activity on HIF-1α activity without adversely affecting cellular viability. Notably, the HIF-1α inhibition resulted in significant reduction in 3D tumorsphere growth indicating a hypoxia related effect of the HIF-1α inhibitors with only a rather moderate reduction in 2D attached cell growth (representing normoxic growth conditions). Notably, and as shown in more detail below, HIF-1α inhibition had also substantial inhibitory effect on expression of HIF-1α, Mct-4, Hexokinase 2, and Pyruvate Dehydrogenase Kinase 1 that would otherwise be observed under hypoxic conditions. Therefore, HIF-1α inhibition also suggests significant potential for metabolic interference, particularly in tumor cells that express HIF-1α and Mct-4. In addition, and as also shown in more detail below, the inventor discovered that expression of a significant number of HIF-dependent genes (including HK2, Hexokinase 2) was substantially inhibited under hypoxic conditions upon exposure of cells to the HIF-1α inhibitors (e.g., NTW-4975). In contrast, HIF-1α inhibition had negligible effects on cells under normoxic conditions, once more indicating therapeutic potential for treatment of tumor cells HIF-1α and Mct-4.

In still further studies, the inventor investigated the effect of Met-4 tumorsphere volume and noted that downregulation of Mct-4 via siRNA reduced tumorsphere volume in a statistically significant manner. Conversely, recombinant overexpression of Mct-4 significantly increase tumorsphere volume, but not in a 2D growth model, thereby implicating Mct-4 as a potential therapeutic target. Still further, the inventor observed that Rab25, HIF-1α, Mct-4, and CD147 were significantly increased in 3D growth as compared to 2D growth.

To further validate Mct-4 as a critical element in tumor growth, the inventor tested if HIF-1α driven growth inhibition of tumorspheres could be reversed by overexpression of Mct-4. Remarkably, overexpression of Mct-4 had a substantial effect on the inhibition of tumorsphere growth in the presence of a HIF-1α inhibitor.

The inventor also sought to ascertain whether or not Mct-4 would have modulating effects on tumor cell invasiveness and tested Panc I cells for TGF-β mediated invasiveness. Notably, upon inhibition of Mct-4 expression, TGF-β mediated invasiveness was abrogated, underscoring once more the therapeutic potential of targeting Mct-4.

Based on the above findings and further data below, the inventor therefore contemplates that therapeutically effective cancer therapy can be performed in tumors that have upregulated (as compared to corresponding normal tissue) HIF-1α and/or Mct-4 expression. Upregulated, as used herein, refers to at least 10%, or at least 20%, or at least 30%, or at least 40%, or at least 50% higher expression compared to a corresponding normal tissue. Viewed from a different perspective, the inventor contemplates that therapeutic efficacy of metabolic inhibitors in the treatment of cancer can be predicted by quantitative analysis of specific markers, and particularly of Mct-4, HIF-1α, Hexokinase-2, and/or CD147. Furthermore, in cases where the tumor cells have an increased expression of these markers (as compared to corresponding non-tumor cells), the tumor can be treated by administration of the metabolic inhibitor, to thereby confer therapeutic effect. Among other options, especially contemplated metabolic inhibitors include a HIF-1α inhibitor, which may be a small-molecule drug, an antibody or fragment thereof, or a drug downregulating expression of HIF-1α. Small molecule drug inhibitors of HIF-1α include Melatonin, NB-5-MT, Manassantin A, Manassantin B, EF-24, Curcumin, Artepillin C, Baccharin, Moracin O, MO-460, AF, Digoxin, EZN-2208, 2-ME2, Echinomycin, Chaetocin, Menadione, Ethacrynic acid, 103D5R, AAL993, AG1478. YC-1, 17-AAG, and AC1-004, as elaborated in Xu R, et al. Action Sites and Clinical Application of HIF-1α Inhibitors. Molecules. 2022 May 26; 27 (11): 3426. doi: 10.3390/molecules27113426. PMID: 35684364; PMCID: PMC9182161 (which is incorporated by reference herein). Contemplated metabolic inhibitors also include mitochondrial targeted inhibitor and/or an inhibitor of HIF-1α interaction with P300. In still further contemplated aspects, suitable metabolic inhibitors further include compounds that will interfere with glycolysis (e.g., 2-deoxyglucose).

As will be readily appreciated, there are various manners of quantitating contemplated markers, and all known manners are deemed suitable for use herein, including quantitating proteins levels (e.g., using mass spectroscopy-based methods, antibody-based methods, etc.), gene expression levels (e.g., rt-qPCR, hybridization methods), etc. In still further contemplated methods, it should be appreciated that the tumor cells can also be analyzed for the presence and/or relative distribution of various metabolites, and especially metabolites that are associated with glycolysis. Viewed from a different perspective, metabolic flux can be ascertained and, where desired, compared with metabolic flux of a non-tumor cell.

Consequently, the inventor also contemplates that cancer (e.g., solid tumors) can be treated using one or more metabolic inhibitors wherein at least one of the inhibitors directly or indirectly targets HIF-1α, and wherein at least one other inhibitor targets a metabolic pathways, and especially the glycolytic pathway. For example, the HIF-1α inhibitor can be an antibody or a small molecule drug (NTW-4975), while the metabolic inhibitor can be a hexokinase-2 inhibitor. In still further contemplated aspects, an antibody against Mct-4 may be used, which may also be coupled to a recombinant T cell or NK (Natural Killer) cell that expresses a chimeric antigen receptor (CAR). In such case, binding of the CAR will mediate a cytotoxic response in the T cell or NK cell, leading to preferential destruction of tumor cells overexpressing Mct-4.

While any suitable NK cell line may be used, as disclosed in more detail herein, the NK-92 cell line is an immortalized cell line suitable for transfection and immunotherapy. Accordingly, the term “NK-92” refers to natural killer cells derived from the highly potent unique cell line described in Gong et al. (1994), rights to which are owned by NantKwest, Inc. (hereafter, “NK-92 cells”). The immortal NK cell line was originally obtained from a patient having non-Hodgkin's lymphoma.

NK-92 cells may be modified e.g., by introduction of exogenous genes. NK-92 cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92, NK-92-CD16, NK-92-CD16-γ, NK-92-CD16-ζ, NK-92-CD16 (F176V), NK-92 MI, and NK-92CI. NK-92 cells are known to persons of ordinary skill in the art, to whom such cells are readily available from NantKwest, Inc. NK-92 cells may be modified to express a chimeric antigen receptor (hereafter, “CAR-modified NK-92 cells”). In some embodiments, the NK-92 cells may express a high affinity CD16 receptor on the cell surface.

The inventor further contemplates administration of a nucleic acid comprising a means of inhibiting expression of Mct-4 or CD147 in the cell. The means may comprise an antisense RNA. The means may be an siRNA, shRNA, or an miRNA. The RNA may be encapsulated for administration. The means could be an antisense DNA, wherein the cellular production of mRNA is targeted, thereby inhibiting the expression of Mct-4 or CD147. The antisense DNA may be encoded by a viral vector. The viral vector may be an Ad5 vector. The Ad5 vector may be lacking the E1 and/or E2b gene regions. The means may also comprise genome editing. The means of genome editing may comprise CRISPR-Cas9, TALEN, or Zinc finger nucleases. Administration of the means for inhibiting expression of Mct-4 or CD147 may comprise subcutaneous administration, ex vivo administration into PBMC derived from patient apheresis product, or direct intratumoral administration.

With regards to viral vectors, it is especially preferred to use viruses already established in cancer therapy, including adenoviruses, adeno-associated viruses, alphaviruses, herpes viruses, lentiviruses, etc. However, among other appropriate choices, adenoviruses are particularly preferred. Moreover, it is further generally preferred that the virus is a replication deficient and non-immunogenic virus, which is typically accomplished by targeted deletion of selected viral proteins (e.g., E1, E3 proteins). Such desirable properties may be further enhanced by deleting E2b gene function, and high titers of recombinant viruses can be achieved using genetically modified human 293 cells as has been recently reported (e.g., J Virol 1998 February; 72 (2): 926-933). Most typically, the desired nucleic acid sequences (for expression from virus infected cells) are under the control of appropriate regulatory elements well known in the art.

### Experimental Data

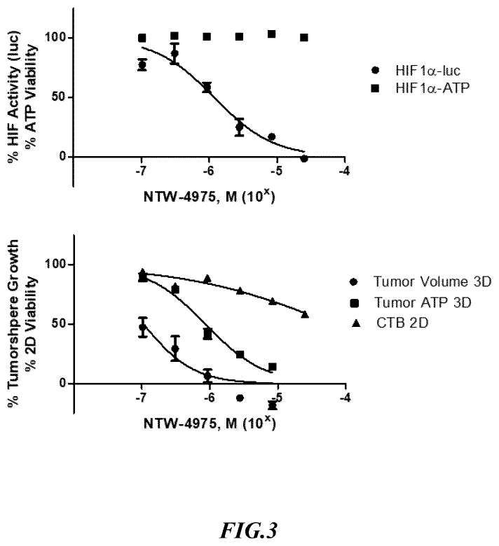

In one set of experiments, NTW-4975 was shown to inhibit HIF-1α activity but not viability (ATP), and reduced tumorsphere growth (3D model) but not growth of attached cells (2D model). Here, Ishikawa cells were seeded into 384 well microplates overnight and exposed to HIF-1α inhibitors for 72 hr. The 3D viability was determined by tumorsphere growth (Celigo) and ATP viability assay (Promega), while 2D growth was determined by CellTiterBlue (Promega), and exemplary results are shown in FIG. 3. As can be readily seen, HIF-1α inhibition affected 3D cells growth to a significantly larger extent than 2D growth.

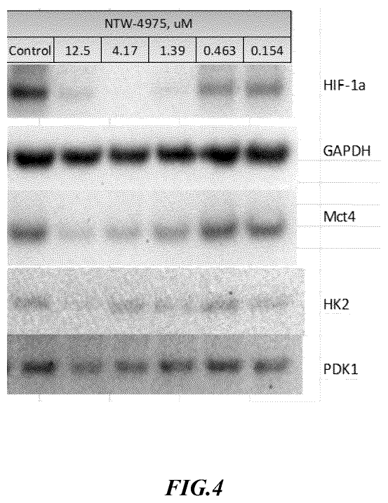

HIF-1α inhibition by NTW-4975 also inhibited hypoxia induced expression of HIF-1α, Mct-4, Hexokinase 2, and Pyruvate Dehydrogenase Kinase 1 in Ishikawa cells. Here, Ishikawa cells were exposed to HIF-1α inhibitors for 24 hr at 1% 02. Lysates were prepared and subjected to SDS-PAGE. Blots were probed with antibodies to HIF-1α (Genetex 127309), Mct4 (sc-376465), HK-2 (CST 2867), PDK1 (Invitrogen MA5-15797) and GAPDH (CST-2118), and exemplary results are shown in FIG. 4.

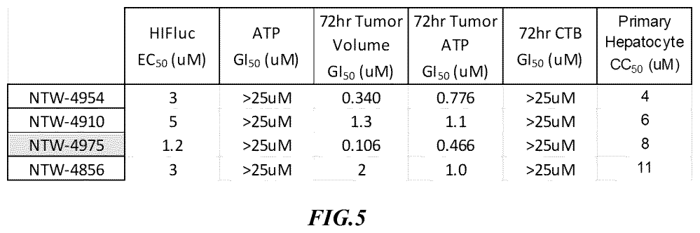

Cell Viability Analysis: A 72 hr viability assay was performed in primary human hepatocytes. Here, primary human hepatocytes in 96 wells (w/matrigel overlay), HepG2 cells, and Ishikawa cells were exposed to HIF-1α inhibitors for 72 hr. HIF-1α activity (OneGlo, Promega) and cell viability (CellTiterGlo, Promega) were determined, and exemplary results are shown in the in Table of FIG. 5.

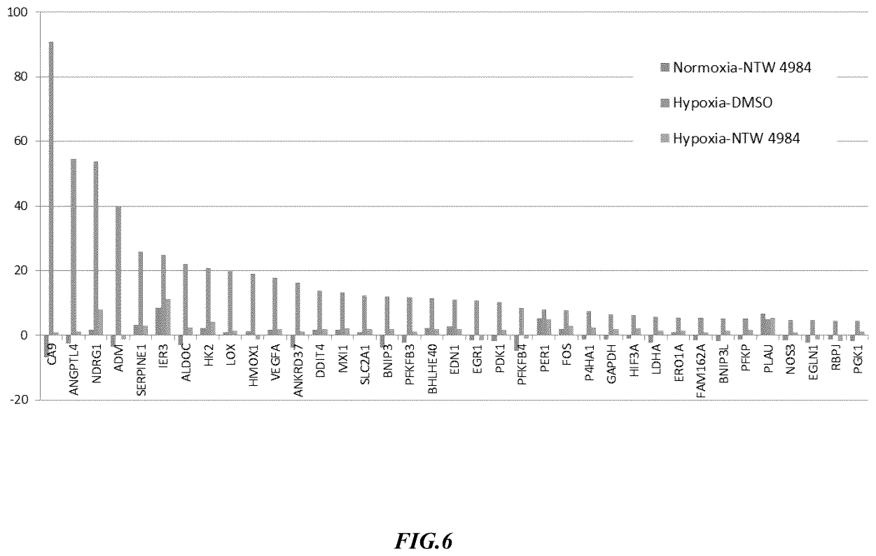

HIF-dependent gene expression: A qPCR array was interrogated using NTW-4984 inhibition of HIF-dependent gene expression. Here, Ishikawa cells were exposed to either normoxia or hypoxia (1% O₂) for 24 hr in the presence and absence of NTW-4984. mRNA was then purified, and cDNA was reverse transcribed and evaluated by a Hypoxia Signaling Pathway PCR Plus Array (SA Biosciences). Exemplary results are shown in FIG. 6.

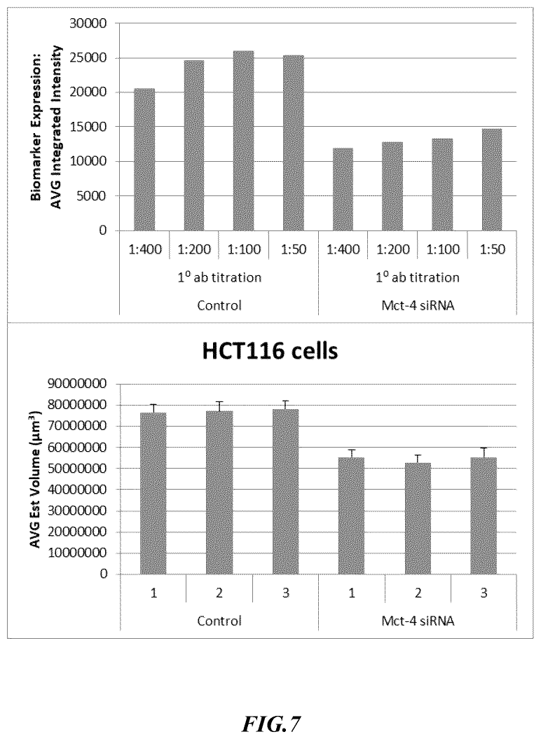

Target Validation: Depletion of Mct4 in HCT116 cancer cells decreased tumorsphere volume (non-attached 3-dimensional growth). FIG. 7, upper panel depicts results for 2K cells/well plated in a flat-bottom 384-well plate after 72 hr in culture. Cells were stained with Alexa-Fluor, and fluorescence image was read on Celigo; The lower panel depicts 2K cells/well plated in a round bottom 384-well plate after 72 hr in culture. Tumorspheres were imaged on Celigo.

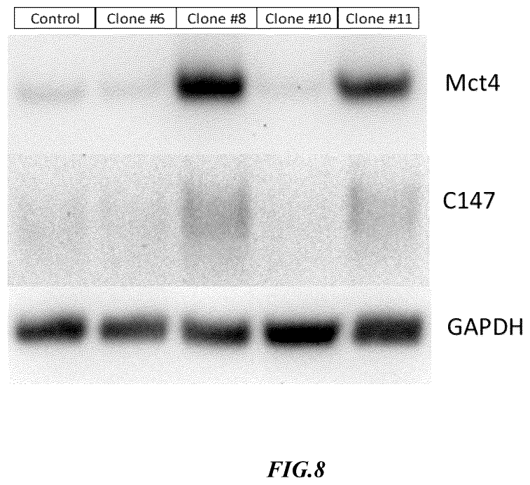

Co-expression of CD147 with Met-4: Stable Clones of Ishikawa cells transfected with Monocarboxylic Acid Transporter 4 were analyzed, and co-expression of CD147 in Mct4 transfected cells was repeatedly observed as can be taken from FIG. 8.

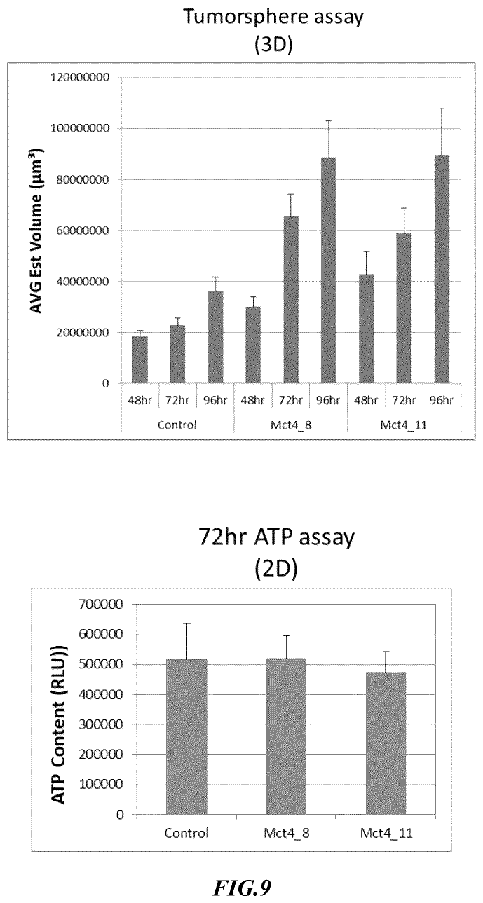

Target Validation: Mct4 overexpression increased tumorsphere volume (non-attached 3-dimensional growth), but not ATP content (cell viability in attached cells) as can be seen from the data in FIG. 9.

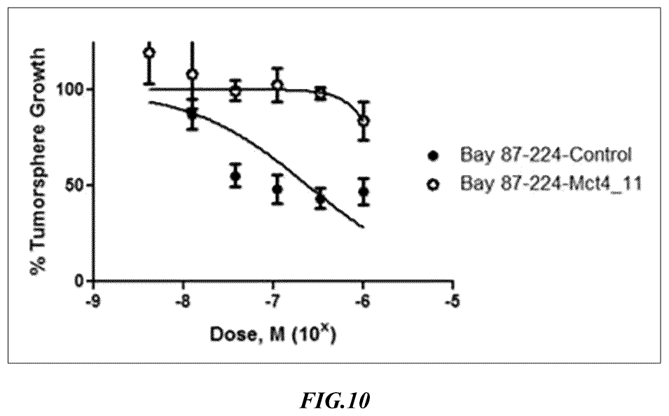

Mct-4 overexpression reverses tumorsphere growth inhibition by HIF-1α inhibitor (Bay87-2243): Here, Ishikawa cells were seeded into 384-well round bottom (tumorsphere), 2000 cells/36 ul/well. After 18 hr, 10× drug was added, and after 72 hr, plates were imaged. Exemplary results are shown in FIG. 10.

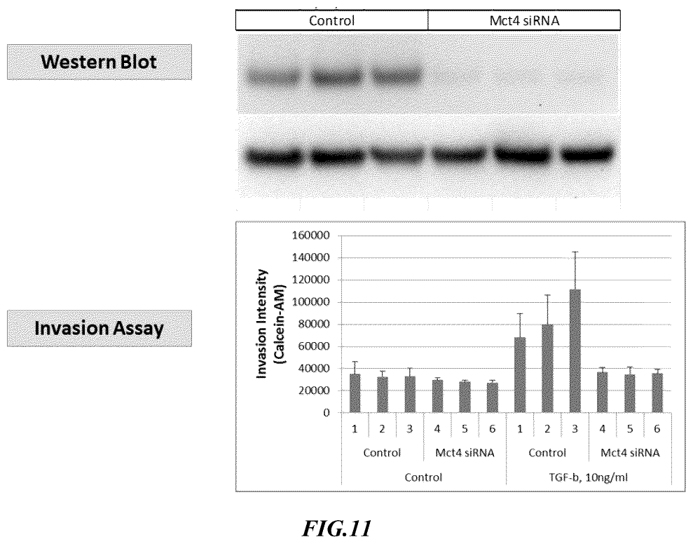

Target Validation: siRNA-mediated depletion of Mct4 inhibited TGF-β driven invasion of Panc1 cells as can be taken from the data in FIG. 11.

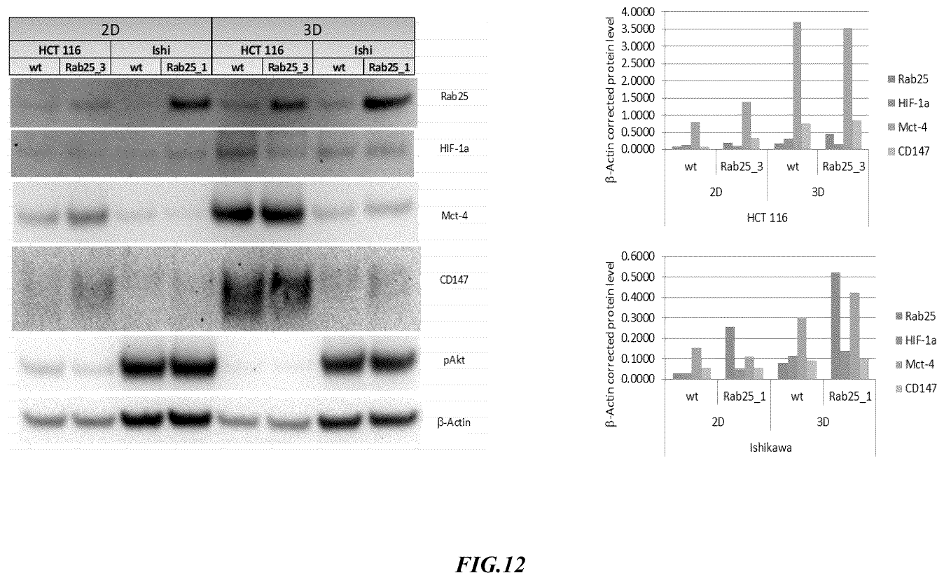

Rab25, HIF-1α, Mct4, and CD147 expression increased in 3D growth format for both HCT116 and Ishikawa cells. Here, Cells (2000/well) were seeded into 384-well low-attachment U-bottom microplates. After 5 days, cells were harvested, pelleted, and lysed along with cells grown in flasks (2D). Both lysates were subjected to Western Blot, and exemplary results are depicted in FIG. 12.

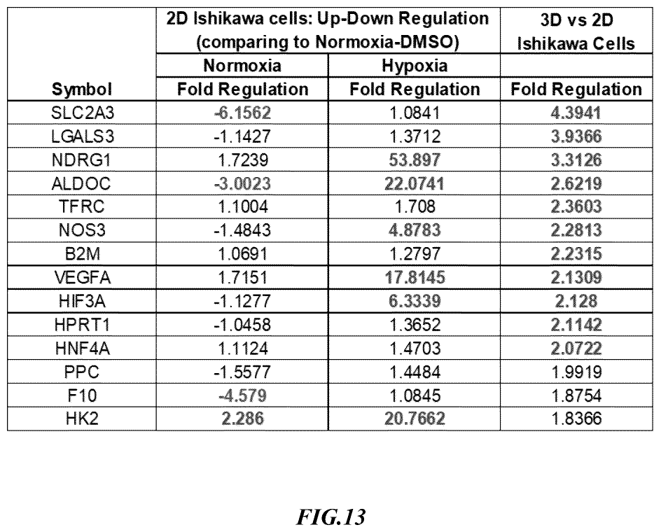

qPCR Profiler showed distinct mRNA transcription between 2D growth under hypoxia vs. 3D growth under normoxic conditions. Here, 2D proliferating Ishikawa cells were exposed to either normoxia or hypoxia (1% O₂) for 24 hr. For 3D proliferating cells, 2000 cells/well were seeded into 384-well low-attachment U-bottom microplates (Nexcelom Biosciences) and harvested after 4 days. mRNA was purified and cDNA was reverse transcribed and evaluated by Hypoxia Signaling Pathway PCR Plus Array (SA Biosciences). Exemplary results are shown in the table of FIG. 13.

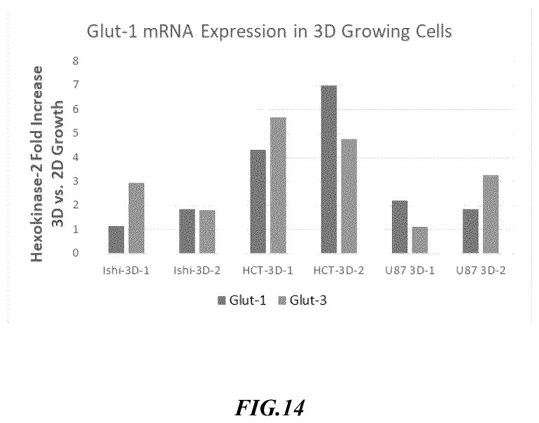

qPCR Profiler showed increased Glut-1 & Glut-3 (Glucose Transporters) mRNA transcription between 2D growth vs. 3D growth, both under normoxic conditions. Here, 2D proliferating Ishikawa cells were exposed to normoxia for 24 hr. For 3D proliferating cells, 2000 cells/well were seeded into 384-well low-attachment U-bottom microplates (Nexcelom Biosciences), maintained at 37° C. under normoxic conditions, and harvested after 4 days. mRNA was purified and cDNA was reverse transcribed and evaluated by Hypoxia Signaling Pathway PCR Plus Array (SA Biosciences). See FIG. 14.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A method of predicting therapeutic efficacy of metabolic inhibitors in the treatment of cancer, the method comprising:
performing quantitative assays for tumor levels of at least one marker selected from the group consisting of monocarboxylate transporter 4 (Mct4), HIF-1α, Hexokinase-2, and CD147; and
administration of the metabolic inhibitor when the tumor expression of the marker is significantly greater than its expression in healthy tissue.

2. The method of claim 1, wherein the metabolic inhibitor is a HIF-1α inhibitor.

3. The method of claim 2, wherein said HIF-1α inhibitor is a mitochondrial targeted inhibitor.

4. The method of claim 2, wherein said HIF-1α inhibitor is an inhibitor of HIF-1α interaction with P300.

5. The method of claim 1, wherein quantitative assays of the tumor levels of the markers are protein levels of the markers, and wherein the protein levels of the markers are compared with corresponding protein levels assayed in normal, non-cancerous tissue.

6. The method of claim 1, wherein said assay measures tumor protein levels.

7. The method of claim 1, wherein said assay measures both tumor protein levels and normal tissue protein levels.

8. The method of claim 1, wherein said assay measures tumor mRNA levels.

9. The method of claim 1, wherein said assay measures both tumor mRNA levels and normal tissue mRNA levels.

10. The method of claim 1, wherein said assay measures tumor metabolic flux.

11. A method of treating solid tumors, the method comprising administering a composition comprising 2-deoxyglucose and an inhibitor of HIF-1α.

12. The method of claim 11, wherein said HIF-1α inhibitor is a mitochondrial targeted inhibitor.

13. The method of claim 11, wherein said HIF-1α inhibitor is an inhibitor of HIF-1α interaction with P300.

14. The method of claim 11, wherein said HIF-1α inhibitor is a small molecule inhibitor.

15. A method of treating solid tumors, comprising administering to a patient a composition comprising a means of binding to Mct4, wherein the means, when bound to Mct4, inhibits growth of Mct4-expressing cells in an in vitro tumorsphere model of cell growth.

16. The method of claim 15, wherein the means is an antibody or binding domain thereof.

17. The method of claim 15, wherein the means is a T cell or a natural killer (NK) cell, wherein the T cell or NK cell comprises a CAR, and wherein the CAR comprises the means for binding Mct4.

18. The method of claim 15, wherein the means comprises an antisense RNA, an siRNA, shRNA, or an miRNA that is targeted to inhibiting the expression of Mct-4 or CD147.

19. The method of claim 15, wherein the composition is administered to a tumor patient.

20. The method of claim 19, wherein the administration is subcutaneous administration, ex vivo administration into PBMC derived from patient apheresis product, or direct intratumoral administration.
